(19) United States
(12) Patent Application Publication
(10) Pub. No.: US 2026/0283909 A1
(43) Pub. Date: Sep. 24, 2026

(54) DENTAL ADHESIVES COMPRISING NANO-HYDROXYAPATITE AND PROPOLIS

(71) Applicant: Imam Abdulrahman Bin Faisal University, Dammam (SA)

(72) Inventors: Maria Salem IBRAHIM, Dammam (SA); Suliman Yousif SHAHIN, Dammam (SA); Ali Ibrahim ALKHALAF, Dammam (SA); Muneer Hani ALSHUYUKH, Dammam (SA); Mohammed Muslim ALHULAMI, Dammam (SA); Abdulaziz Saeed ALAMRI, Dammam (SA)

(73) Assignee: Imam Abdulrahman Bin Faisal University, Dammam (SA)

(21) Appl. No.: 19/086,817

(22) Filed: Mar. 21, 2025

(51) Int. Cl. A61K 6/30; A61C 7/16; A61C 13/15; A61K 6/17; A61K 6/838; A61K 6/887

(52) U.S. Cl. CPC A61K 6/30; A61C 7/16; A61C 19/003; A61K 6/17; A61K 6/838; A61K 6/887

(57) ABSTRACT

Dental adhesives having re-mineralization and antimicrobial properties including propolis, calcium hydroxyapatite nanoparticles and an orthodontic adhesive resin. The dental adhesive demonstrates a high shear bond strength and a surface hardness which can be attributed to the use of nanohydroxyapatite particles. Additionally, the incorporation of propolis contributes to the anti-microbial properties of the adhesive which minimizes the risk of white spot lesion formation after orthodontic treatments. A method of bonding an orthodontic device to a tooth using the dental adhesives of the present disclosure is provided.

### BACKGROUND

### Technical Field

The present disclosure is directed to dental adhesives having antimicrobial and remineralization properties. More particularly, the present disclosure relates to dental adhesives based on propolis and hydroxyapatite nanoparticles that can be used in various dental restorative procedures.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Dental adhesives are an essential component in modern dentistry, facilitating the bonding of restorative materials to tooth structures. They play a critical role in ensuring the longevity and success of various dental treatments, including fillings, crowns, bridges, and veneers. The type of adhesive and its method of application is chosen based on the anatomy of tooth and the type of dental appliance to be attached on the tooth. A tooth is typically composed of different layers, including enamel, dentin, and the pulp chamber. The adhesive needs to effectively bond with the enamel and penetrate the dentin layer to strengthen the bond. Conventional dental adhesives usually comprise a mixture of resin monomers, solvents, curing agents, and optionally, inorganic fillers. Dental adhesives can be classified based on their method of application as etch-and-rinse adhesives and self-etch adhesives. In etch-and-rinse systems, a tooth is first etched using an acid which is typically followed by rinsing and drying the tooth. The adhesive is then applied on the tooth to fix a dental appliance on the tooth. However, this method requires a careful moisture control during application to prevent complications such as postoperative sensitivity. Self-etch adhesive systems integrate the etching and bonding steps into a single application. This method simplifies the bonding process and reduces sensitivity issues associated with traditional etching methods. Self-etch adhesives can also be applied in a single or multiple steps. However, the bond strength in self-etch systems is relatively lower than in etch-and-rinse systems. Moreover, self-etch systems require more precision in application to achieve desired results.

Existing adhesives often face challenges such as microleakage, sensitivity to moisture during application, and limited long-term durability. Recent innovations in dental adhesives are more focused on their bonding efficacy, biocompatibility, and durability. New and improved materials are being used in the developments of adhesives such as incorporation of bioactive materials, use of nanofillers such as nanosilica and polymerizable monomers along with polymerization initiators for curability. Another problem that is frequently faced by orthodontic patients is demineralization of enamel that is visible as white spot lesions at the areas of teeth where an orthodontic appliance is fixed. Moreover, fixed dental appliances including brackets and wires can trap food particles and microbes, leading to the accumulation of plaque. Hence, it is essential to develop innovative solutions to prevent and manage enamel demineralization in patients undergoing orthodontic treatments.

US20030219388A1 describes dental adhesive films that assist in the remineralization of teeth that have been damaged by caries, enamel lesions, or open dentine channels. The active compound in the film is a calcium salt of phosphates, fluorides, fluorophosphates, or their mixtures which may optionally contain hydroxyl, carbonate, or chloride ions. The calcium salt may be of hydroxyapatite, fluoroapatite or their mixtures.

U.S. Ser. No. 11/179,297B2 describes antimicrobial polymeric compositions comprising metal-containing methacrylates, such as calcium methacrylate, tin methacrylate, copper methacrylate, and silver methacrylate. The methacrylates are incorporated into a curable organic matrix and further comprise an inorganic filler, a radiopacifier, a polymerization initiation system, an inhibitor of polymerization and optical modifiers. The compositions may also comprise natural extracts including essential oils of copaiba, butii, vitis, cardol, cardenol, oregano, aroeira, garlic and propolis.

EP0900560A1 describes a tooth composite comprising shellac dissolved in alcohol, at least one of antibacterial constituent, anti-bacteria antibody, and efficacious constituent. The anti-bacterial constituent includes at least one of propolis, hinokitiol and chitin chitosan; and the anti-bacteria antibody includes anti-dental-caries-bacteria antibody and/or anti-periodontal-disease-bacteria antibody. To enhance color tone, the composite may contain pigments, one of which may be hydroxyapatite.

U.S. Pat. No. 8,815,973B2 describes a dental cement comprising a polymerizable resin as the first component and a second component comprising more than one type of amines selected from the group consisting of primary amine, secondary amines, tertiary amines and quaternary amines or a compound having a suitable mercapto (SH—) group, a hindered amine or a dimethylthiotoluenediamine (DMTDA). The cement product may optionally comprise one or more fillers wherein one of the fillers can be hydroxyapatite.

Nanomaterials and natural substances are increasingly being used in the formation of dental adhesives owing to their improved bond forming capabilities and antimicrobial activities. Hydroxyapatite based composites have been hypothesized to have improved mechanical properties (See: Mehdi Sadat-Shojai et. Al. Hydroxyapatite nanorods as novel fillers for improving the properties of dental adhesives: Synthesis and application. Dental materials, Volume 26, Issue 5, May 2010; Alia J. Rubaie, Karen A. Schulze. Polymer chemistry of dental adhesives. Pacific Journal of Health, Vol 1, Issue 1 (2018); Yohann Catel et. al. Synthesis and evaluation of new phosphonic, bisphosphonic and difluoromethylphosphonic acid monomers for dental application. European Polymer Journal, Volume 48, Issue 2, February 2012, Pages 318-330). Additionally, polyphenols and propolis are being evaluated for their bond forming abilities and bonding durabilities as adhesive components (See: ICC de Moraes Porto et. al. Polyphenols and Brazilian red propolis incorporated into a total-etching adhesive system help in maintaining bonding durability. Heliyon. 2021 Feb. 19; 7(2):e06237). Further, synergistic combinations of various compounds can be explored for developing advanced and more effective dental adhesives. (See: L Grenho, J Barros, C Ferreira, V R Santos, F J Monteiro, M P Ferraz, M E Cortes. In vitro antimicrobial activity and biocompatibility of propolis containing nanohydroxyapatite. Biomed Mater. 2015 Mar. 18; 10(2):025004).

Each of the aforementioned dental adhesives either lack sufficient antibacterial properties or may not effectively promote remineralization, both of which are required for a good oral hygiene and optimal dental treatment outcomes.

Accordingly, it is one object of the present disclosure to provide dental adhesives having both antimicrobial and remineralization properties. Another object of the present disclosure is to provide adhesives comprising propolis and hydroxyapatite nanoparticles. A synergistic combination of these materials provides dental adhesives with a high bond strength, enhanced protection against microbial activities and reduced risk of demineralization resulting from the use of fixed dental appliances.

### SUMMARY

In an exemplary embodiment, a dental adhesive is described. The dental adhesive includes propolis in an amount of about 4 wt. % to about 6 wt. %; calcium hydroxyapatite nanoparticles in an amount of about 1.5 wt. % to about 2.5 wt. %; and the balance an orthodontic adhesive resin, wherein wt. % is based on a total weight of the dental adhesive.

In another exemplary embodiment, a method of bonding an orthodontic device to a tooth is described. The method includes comprising applying a layer of an orthodontic adhesive to the tooth and/or the orthodontic device, wherein the orthodontic adhesive comprises an propolis in an amount of about 4 wt. % to about 6 wt. %, calcium hydroxyapatite nanoparticles in an amount of from about 1.5 wt. % to about 2.5 wt. %; and the balance an orthodontic adhesive resin, wherein wt. % is based on a total weight of the dental adhesive; and bonding the orthodontic device to the tooth by pressing the orthodontic device against the tooth with the orthodontic adhesive between the tooth and the orthodontic device.

In another exemplary embodiment, a method of making a dental adhesive composition is described. The method includes mixing an amount of propolis of about 4 wt. % to about 6 wt. % and an amount of calcium hydroxyapatite nanoparticles in an amount of about 1.5 wt. % to about 2.5 wt. % into an orthodontic adhesive resin, wherein wt. % is based on a total weight of the dental adhesive composition.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic flowchart of a method of bonding an orthodontic device to a tooth, according to certain embodiments.

FIG. 2 is a pictorial image depicting various compositional groups, each having different adhesive components, according to certain embodiments.

FIG. 3A is a schematic illustration depicting the process steps for shear bond strength assessment, according to certain embodiments.

FIG. 3B shows a histogram representing shear bond strengths of different adhesive samples, according to certain embodiments.

FIG. 4A is a schematic illustration depicting the process steps for surface hardness assessment, according to certain embodiments.

FIG. 4B is a graph representing mean standard deviations of surface hardness % (% SH) of different adhesive samples after exposure to demineralization for 2 days, according to certain embodiments.

FIG. 5 is a schematic illustration depicting the process steps for microbiological assessment, according to certain embodiments.

FIG. 6 shows results of colony forming units (CFU) test for Streptococcus mutans (S. mutans) biofilms per adhesive sample, according to certain embodiments.

FIG. 7 shows mean standard deviation of S. mutans biofilm metabolic activity for each adhesive sample after 48 hours as measured by 3-[4,5-dimethylthiazol-2-yl]-2,5 diphenyl tetrazolium bromide (MTT) assay, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

As used herein, “orthodontic appliance” refers to a device used to correct misaligned teeth and jaws.

As used herein, “demineralization” refers to the loss of minerals from the tooth enamel, often leading to decay and white spot lesions.

As used herein, “nanoparticles” refers to particles whose size ranges between 1 to 100 nm.

As used herein, “nanohydroxyapatite” refers to a nanoparticle form of hydroxyapatite.

As used herein, “dental adhesive” refers to a bonding agent used to fix a dental device to a tooth.

As used herein, “restorative procedure” or “dental restoration” refers to a treatment process that repairs or replaces damaged or missing teeth to restore their function and appearance.

As used herein, “particle size” may be considered the lengths or longest dimensions of a particle.

As used herein, “dentin” or “dentine” refers to a tissue beneath the enamel that forms the bulk of a tooth and surrounds the pulp.

Fixed orthodontic appliances pose a significant risk of the accumulation of dental plaque and magnify the challenge to achieve a proper oral hygiene. Moreover, due to a continuous use of these fixed appliances, enamel demineralization occurs that appears as white spot lesions on the teeth of a person undergoing orthodontic treatment. These lesions are aesthetically unpleasant and may cause further destruction of tooth structure. Accordingly, aspects of this disclosure are directed to dental adhesives having antimicrobial and remineralization properties.

According to an aspect, the dental adhesive of the present disclosure includes fillers including propolin and a nanohydroxyapatite, mixed with an orthodontic adhesive resin wherein the composition promotes oral health and the natural repair processes of the tooth structure. Propolin (also referred to as propolis) has anti-microbial and anti-inflammatory properties making it effective in preventing oral infections. Nano-hydroxyapatite helps in remineralization and enamel repairing and an adhesive resin provides a strong bonding required to attach a dental appliance to a tooth.

Hydroxyapatites are composed of calcium and phosphorus. Due to their composition being similar to the human bones and teeth, hydroxyapatites are often preferred in dental repairing functions including re-mineralization of teeth. In an aspect, nanoparticles of hydroxyapatites are preferred. Nanoparticles provide better bioactivity and are resorbed readily hence, the hydroxyapatite nanoparticles are more effective in dental restoration and dental care. Both pure and modified hydroxyapatites may be used in the dental adhesives of the present disclosure. Modified hydroxyapatites include ion substituted hydroxyapatites wherein the ions include any of fluoride, magnesium, strontium and zinc incorporated into the hydroxyapatite lattice structure. A non-limiting example of hydroxyapatites that may be used in the dental adhesives is calcium hydroxyapatite since calcium hydroxyapatite is naturally present in the tooth enamel. The size and shape of the hydroxyapatite nanoparticles is selected to resemble the size and shape of the hydroxyapatite particles naturally present in the enamel. Accordingly, the hydroxyapatite nanoparticles may have a particle size ranging from 30 nm to 100 nm. In an aspect, hydroxyapatite nanoparticles having a particle size in the range of 30 nm to 60 nm is preferred.

In one aspect, the dental adhesive includes an amount hydroxyapatite nanoparticles in an range of about 1.5 wt. % to about 2.5 wt. % based on a total weight of the dental adhesive. In some aspects, dental adhesive includes hydroxyapatite nanoparticles in a range of 1.6 wt. % to 2.4 wt. %, preferably in a range of 1.7 wt. % to 2.3 wt. %, preferably in a range of 1.8 wt. % to 2.2 wt. %, in a range of 1.9 wt. % to 2.1 wt. %, and/or 1.9 wt. % to 2.0 wt. %, where wt. % is based on a total weight of the dental adhesive. In an aspect, hydroxyapatite nanoparticles in an amount of about 2 wt. % based on a total weight of the dental adhesive are preferred.

In a preferred embodiment of the invention, the dental adhesive further comprises particles of a high molecular weight polyolefin wax. In aspects of the present disclosure, the polyolefin wax particles provide an aggregating site for propolis in the dental adhesive. Upon application to a substrate, polyolefin wax particles exhibit a tendency to migrate towards the surface of the dental adhesive, thereby increasing the concentration of propolis at the surface of the dental adhesive. The polyolefin wax particles typically slough off the cured dental adhesive surface leaving behind the propolis as a component of the cured dental adhesive matrix.

The polyolefin wax is preferably a high molecular weight polyethylene wax. For example, the polyethylene wax may have a weight molecular weight of from 20,000 to 200,000, preferably 30,000-150,000, 40,000-100,000 or about 60,000. The wax particles are preferably present in an amount that is no greater than the total amount by weight of the propolis. Preferably, the wax particles are present in an amount by weight that is less than the total amount by weight of propolis, such as a propolis:wax weight ratio of 1:0.9, 1:0.8, 1:0.5, or 1:0.2. The polyolefin wax particles may be larger than the hydroxyapatite nano particles in the dental adhesive. The polyolefin wax particles may have an average particle size in the range of 50 nm-50 μm, 100 nm-25 μm, 500 nm-20 micron, or 1,000 nm-10 μm.

The dental adhesive further includes an antimicrobial compound, preferably having a natural origin. In an aspect, the antimicrobial compound is propolis. Propolis is a natural substance produced by bees from the resin of trees and plants and is used by the bees to seal and protect their hives. Propolis is found to be rich in bioactive compounds such as flavonoids, phenolic acids, esters and polyphenols and acts as a natural shield against infections and inflammation. These bioactive compounds have been shown to promote healing and tissue regeneration in patients undergoing orthodontic treatments. In one aspect, the dental adhesive includes propolis in an amount of about 4 wt. % to 6 wt. % based on a total weight of the dental adhesive. In some aspects, propolis is present in an amount in a range of about 4.2 wt. % to 5.8 wt. %, preferably in a range of 4.4 wt. % to 5.6 wt. %, in a range of 4.6 wt. % to 5.4 wt. %, in a range of 4.8 wt. % to 5.2 wt. %, and/or in a range of 4. wt. % to 5 wt. %. In an example, propolis is present in an amount of about 5 wt. % based on a total weight of the dental adhesive.

The dental adhesives further include an adhesive for bonding the dental materials to a tooth. The adhesive is an orthodontic adhesive resin. Suitable orthodontic adhesives that may be used in the dental adhesives of the present disclosure include composite resins and glass ionomer cements. Composite resins are primarily based on a polymer matrix incorporated with filler particles to enhance their mechanical properties. The polymer matrix of these resins is mainly composed of methacrylate monomers. A non-limiting example of polymer matrix is a matrix based on bisphenol A glycidyl methacrylate (bisGMA). Other methacrylate monomers including diethylene glycol dimethacrylate (DEGMA) and triethylene glycol dimethacrylate (TEGDMA) may be added to the bisGMA composites to reduce viscosity. Once the polymer and the filler particles are combined, the resin is subjected to curing. Curing can be carried out in the presence of light, heat or curing agents, such as acrylates, isocyanates, amines, peroxides and the likes.

In some aspects, dental cements based on glass ionomers can be used as orthodontic adhesive resin. Glass ionomer cements (GICs) are composed of a silicate glass powder and a water-soluble polymer. In an example, a glass ionomer cement is obtained by chemical reaction between a silicate glass powder such as calcium aluminofluorosilicate glass and polyacrylic acid. The reaction results in a powdered cement in which glass particles are encased within a fluoride-rich matrix. GICs release fluoride over time which helps to prevent tooth decay and strengthen the surrounding enamel. The glass ionomer cements further include resin modified glass ionomer cements wherein light-curable resin components are added to silicate glass powder and water-soluble polymer. Addition of resin components to GIC results in composites which are resistant to moisture, release fluoride for a longer time period and have better mechanical properties than GIC.

The dental adhesive comprises orthodontic adhesive resin in an amount of about 91 wt. % to about 95 wt % based on a total weight of the dental adhesive. In an aspect, the dental adhesive comprises orthodontic adhesive resin in an amount in a range of about 91.5 wt. % to about 94.5 wt. %, preferably 92 wt. % to 94 wt. %, 92.5 wt. % to 93.5 wt. %, and/or 92.5 wt. % to 93 wt. % based on a total weight of the dental adhesive. In a non-limiting example, the dental adhesive includes propolis in an amount of about 5 wt. %, calcium hydroxyapatite nanoparticles in an amount of about 2 wt. % and an orthodontic adhesive resin in an amount of about 93 wt. % based on a total weight of the dental adhesive. In another example, the dental adhesive includes propolis in an amount of about 6 wt. %, calcium hydroxyapatite nanoparticles in an amount of about 2.5 wt. % and an orthodontic adhesive resin in an amount of about 91.5 wt. % based on a total weight of the dental adhesive.

A method of preparation of the dental adhesive is further described. The orthodontic adhesive resin is taken as the base matrix for the dental adhesive. In an aspect, methacrylate monomers along with a filler and an initiator may be used as the orthodontic adhesive resin. In an example, light-cured adhesive resin based on methacrylate is selected as the base matrix for the dental adhesive wherein bisGMA monomers are added to another methacrylate monomer such as 2-Hydroxyethyl Methacrylate (HEMA). An initiator compound is added to the mixture of monomers to start the polymerization process when exposed to light. Suitable examples of photoinitiators include camphorquinone, benzophenone phenanthrenequinone and the like. The components are continuously stirred for a uniform mixing followed by the addition of Propolis. Propolis is added gradually to the orthodontic adhesive resin in an amount of about 4 wt. % to about 6 wt. % based on a total weight of the dental adhesive composition. The mixing may be performed manually by a stirrer and the solution may be constantly stirred while being mixed. Once propolis is thoroughly mixed, the hydroxyapatite nanoparticles are introduced into the mixture. The mixture is again continuously stirred to uniformly distribute the nanoparticles throughout the adhesive resin.

In another example, glass ionomer cements are used as the orthodontic adhesive resins wherein a glass powder and a polyacrylic acid solution are mixed in a proportion of 1:1 to 3:1 by weight while being constantly stirred. An example of glass powder is fluoro-aluminosilicate that contains ions such as strontium, calcium and lanthanum. The setting process begins as soon as the components are mixed. At this step, propolis is added gradually to the orthodontic adhesive resin in an amount of about 4 wt. % to about 6 wt. % based on a total weight of the dental adhesive composition. The mixing involves constant stirring which can be done manually by a stirrer. A continuous stirring is preferable to ensure homogenous dispersion of propolis within the resin matrix. This is followed by the gradual addition of the hydroxyapatite nanoparticles to the mixture while being constantly stirred. Stirring also prevents the formation of air bubbles in the mixture which, if not removed, may reduce the strength of the resin. Once prepared, the dental adhesive resin may be subjected to curing in the presence of light. The cured resin may then be tested for desired properties like bonding strength, durability, and bio-compatibility.

Incorporation of propolis and hydroxyapatite nanoparticles into dental adhesives shows a significant remineralization effect while imparting a high shear bond strength to the adhesives. Shear bond strength is a measure of an adhesive's ability to hold bonded surfaces together under shear stress. The exemplary dental adhesive of the present disclosure comprises propolis in an amount of about 5 wt. %, calcium hydroxyapatite nanoparticles in an amount of about 2 wt. % and an orthodontic adhesive resin in an amount of about 93 wt. %, based on a total weight of the dental adhesive, was assessed for the desired properties. In an aspect, the said dental adhesive has a shear bond strength of about 15.7 Mpa. Measuring the surface hardness of a dental adhesive is another parameter of evaluating the strength and durability of the adhesive. The dental adhesives, as described herein, exhibit a surface hardness 60% higher than that of a dental adhesive lacking propolis and hydroxyapatite nanoparticles.

The antimicrobial activity of the dental adhesives was assessed from colony forming unit (CFU) tests which were performed in triplicate to ensure that the results were reliable and statistically significant. A colony-forming unit (CFU) is a unit which estimates the number of microbial cells (bacteria, fungi, viruses etc.) in a sample that are viable and able to multiply via binary fission under the controlled conditions. The CFU test for the dental adhesive had an amplitude of about 7 based on a log₁₀ scale when compared to an adhesive lacking propolis and hydroxyapatite nanoparticles which had an amplitude of about 8 based on a log₁₀ scale. Thus, the amount of bacteria formed on the commercial adhesive sample was about 10 times greater than the amount of bacteria formed on the dental adhesive of the present disclosure.

The dental adhesives were further exposed to Streptococcus mutans biofilm for a period of about 48 hours. A lower mean standard deviation means the adhesive consistently performs well in reducing bacterial activity. The mean standard deviation exhibited by the dental adhesives is as low as about 0.04, or one four-hundredth, of S. mutans biofilm metabolic activity which shows the strong antimicrobial properties of the present dental adhesives.

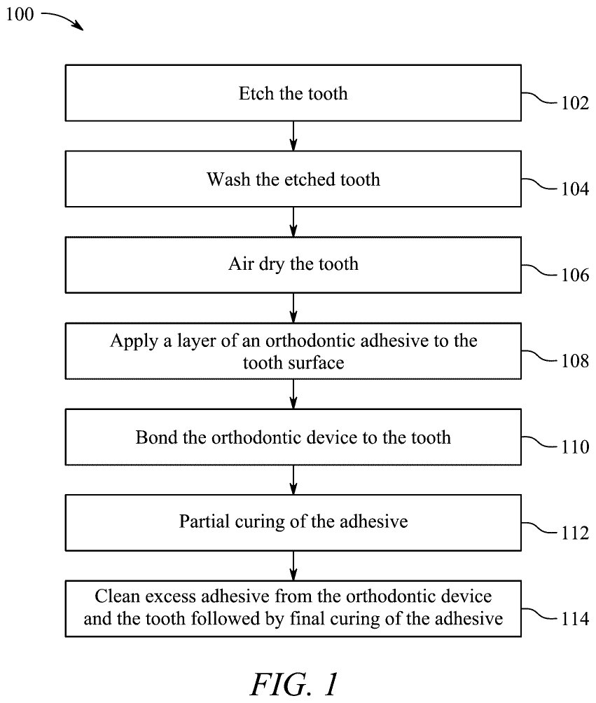

Referring to FIG. 1, a schematic flow chart of a method 100 for bonding an orthodontic device to a tooth is illustrated, according to an aspect of the present disclosure. The order in which the method 100 is described is not intended to be construed as a limitation, and any number of the described steps can be combined in any order to implement the process 100. Additionally, individual steps may be removed or skipped from the process 100 without departing from the spirit and scope of the present disclosure.

At step 102, the method 100 includes etching the tooth before applying the layer of an orthodontic adhesive to the tooth. The method includes using an acidic gel to roughen up the tooth's surface to make it more receptive to the adhesive. Etching may include etching of the enamel, etching of the dentin or etching of both enamel and dentin on the tooth. Etching of the enamel is preferably done to create a porous surface that improves the bond strength of adhesives and restorative materials. Etching of dentin may be performed in restorative procedures where bonding to the dentin is required. Suitable acidic gels that may be used for etching a tooth include gels of hydrofluoric acid, citric acid, polyacrylic acid, nitric acid, maleic acid, phosphoric acid and the like. In an aspect, phosphoric acid gel for etching the enamel and dentin is preferred. The etching may be performed for a time period of about 20 to 40 seconds. In an aspect, the etching may be performed for a period of about 22 to 38 seconds, about 24 to 36 seconds, about 26 to 34 seconds, about 28 to 32 seconds. Etching the tooth for a period of about 30 seconds is preferred.

At step 104, the method 100 includes washing the etched tooth with water. Washing with water is crucial to wash away the acidic residues on the tooth. This step includes rinsing the tooth gently with a water spray. The water may be distilled water, double distilled water or deionized water. In an aspect, the water may be deionized water (dH₂O). Rinsing of the tooth may be carried out for a period of about 25 to 40 seconds. In some aspects, rinsing of the tooth may be carried out for a period of about 30 to 35 seconds. The tooth may be rinsed with water, preferably for a period of about 30 seconds.

At step 106, the method 100 includes air drying the tooth. This is a critical step as moisture on the tooth can interfere with the adhesive's ability to bond properly to the tooth. The tooth may be dried with clean and dry air. An air compressor may be used to provide clean air. The air pressure may be low or moderate such that it does not cause discomfort to the patient undergoing treatment. Air drying of the tooth may be carried out for a period of about 10 to 15 seconds or more till the tooth surface is completely dry.

At step 108, the method 100 includes applying a layer of an orthodontic adhesive to the tooth surface. The orthodontic adhesives are of the compositions as described above and may be applied directly to the tooth and/or to an orthodontic device to be bonded to the tooth. The applied layer of adhesive may be thin or thick depending on the method of application and type of adhesive used. Additionally, the adhesive may be applied in one or more layers if required for an effective bonding. In certain aspects, the adhesive is applied in a thin, even layer on the entire bonding surface for a strong and uniform bonding. Suitable devices may be used for the application of the orthodontic adhesive including a syringe, squeeze tubes and containers. Preferably, a syringe may be used as it allows to dispense a specified amount of adhesive on the desired surface. A brush or an adhesive tip applicator may then be used to spread the adhesive evenly on the bonding surface.

At step 110, the method 100 includes bonding the orthodontic device to the tooth. The orthodontic device may be pressed against the tooth such that the adhesive is between the device and the tooth. For accurately placing the device, the device is gently pressed for a few seconds, allowing the adhesive to evenly distribute between the device and the tooth. Pressing with light pressure helps to remove voids or bubbles that may form at the edges of adhesive while pressing the adhesive on the tooth.

The orthodontic adhesives of the present disclosure are suitable for the attachment of fixed orthodontic appliances to the tooth. In an example, the orthodontic device may be an orthodontic bracket. Other devices such as molar tubes and metal bands may also be attached to the teeth using the present orthodontic adhesives.

After bonding the orthodontic device to the tooth, the adhesive is cured in the presence of light at step 112. Curing hardens the adhesive on the tooth surface. The adhesive layer is exposed to the curing light, which may be a visible light, one or more light emitting diodes (LED) curing lights or a laser. In an example, an LED curing light may be used to cure the adhesive. The method includes partial curing of the adhesive followed by final curing. In partial curing, the adhesive is exposed to curing light for a period of about 5 seconds to about 15 seconds. In certain aspects, the adhesive is partially cured for a period of about 8 seconds to about 12 seconds. In another aspect, the adhesive is partially cured for a period of about 10 seconds.

At step 114, the method 100 includes cleaning the excess adhesive from the orthodontic device and the tooth. Excess adhesive that may squeeze out while pressing the adhesive between the device and the tooth may be removed using scalers. Other instruments such as curettes, pliers and burs may also be used to remove excess adhesive gently without scratching the tooth surface. Step 114 is followed by final curing of the dental adhesive in the presence of light. Final curing results in permanent setting of the adhesive on the tooth surface. The curing at this step can be done in the presence of visible light, a light emitting diode (LED) curing light or a laser. Preferably, the dental adhesive is cured in the presence of the LED curing light. The final curing may be carried out for a longer time than the partial curing step. The final curing time may be performed in a range about 30 to about 50 seconds. In an aspect, the final curing time may be preferably in a range of about 35 to about 45 seconds, and/or 38 seconds to 40 seconds. In an example, the final curing time may be about 40 seconds for the adhesive to harden and fix the orthodontic device on the tooth surface.

### EXAMPLES

The following examples demonstrate the dental adhesives of the present disclosure and assessments for their re-mineralizing and antimicrobial properties as described herein. The examples are provided solely for illustration and are not to be construed as limitations of the present disclosure, as many variations thereof are possible without departing from the spirit and scope of the present disclosure.

### Example 1: Preparation of Experimental Groups

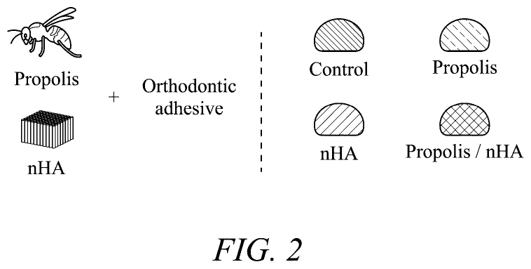

The experiments included four groups as described in Table 1. The first group is a commercial control group formed of 3 M Transbond XT adhesive group, a second group comprises Transbond XT+5 wt. % propolis, a third group comprises Transbond XT+2 wt. % Nano-hydroxyapatite (nHA) and a fourth group comprises Transbond XT+5 wt. % propolis+2 wt. % nHA (Propolis/nHA) group. The propolis group adhesive was prepared by mixing (by weight) the commercial adhesive (Transbond™ XT, 3 M Unitek, USA) with 5% propolis (Nature's Answer®, USA). For the nHA group, 2% hydroxyapatite (HAP01-60, EPRUI Biotech Co., Ltd., China) was added to the Transbond XT adhesive. In the propolis/nHA adhesive group, 5% propolis and 2% nHA were added to the Transbond XT adhesive. All four groups are depicted in FIG. 2.

TABLE 1
Experimental Groups

| Groups | Composition | Abbreviation |
|---|---|---|
| Control | Transbond XT | Control |
| Propolis | Transbond XT + 5 wt. % Propolis | Propolis |
| nano-Hydroxyapatite | Transbond XT + 2 wt. % nano-Hydroxyapatite | nHA |
| Propolis/nano- | Transbond XT + 5 wt. % Propolis + 2 wt. % | Propolis/nHA |
| Hydroxyapatite | nano-Hydroxyapatite | |

### Example 2: Teeth Collection

Extracted human premolars free from caries were used. The teeth extracted for treatment purposes were collected after obtaining verbal consent from patients who were willing to donate the teeth for research purposes. The teeth were stored in 0.01% (w/v) thymol solution (pH 7.0) at 4° C. until use.

### Example 3: Shear Bond Strength (SBS) Assessment

The protocol for bracket bonding as suggested by Sodagar et al. (“Evaluation of the Effect of Propolis Nanoparticles on Antimicrobial Properties and Shear Bond Strength of Orthodontic Composite Bonded to Bovine Enamel”, published by Frontiers in Dentistry on Apr. 30, 2019, incorporated herein by reference in its entirety) was followed. The teeth were cleaned, washed and dried. The teeth were randomly assigned to the different experimental groups (n=10). The teeth were mounted into acrylic resin. Thirty seven percent phosphoric acid gel (Supraetch, manufactured by R&S, Paris, France) was placed on the buccal surfaces of the teeth for 30 seconds for the etching process, and then the teeth were washed with water for 30 seconds and air-dried. A uniform layer of bonding (Single Bond Universal Adhesive, manufactured by 3 M ESPE, Hartford City, Indiana, U.S.A.) was placed on the etched surfaces for all teeth and exposed to light curing for 10 seconds. The different adhesive groups were then placed on a base of standard edgewise 0.022-inch slot stainless steel brackets (3M ID 70202179563, Unitek Gemini Metal Brackets, manufactured by 3 M Unitek, Hartford City, Indiana, U.S.A.) and were seated in the middle part of the crown along the long axis of the tooth. Excess adhesive was cleaned and removed before light curing for 40 seconds. After curing, the samples were kept in a 37° C. incubator for 24 hours before testing. A universal testing machine (Instron 5965, manufactured by Instron Corp. Norwood, Massachusetts, U.S.A) was used to test the SBS. The samples were mounted and set parallel to the vector of the applied force with a load of 5 kN at a constant speed of 0.5 mm/min until the brackets detached. The bonding force was recorded in Newtons (N), followed by dividing the bracket's base area (mm²) to determine the SBS in Megapascal (MPa).

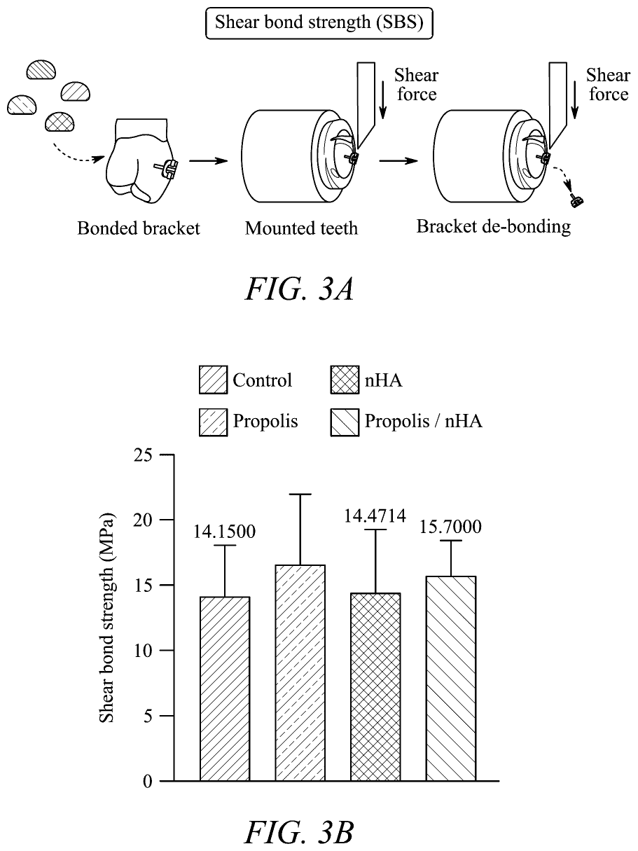

The method for measuring shear bond strength is illustrated in FIG. 3A. As shown, each of the four compositions were used to bond a bracket to a tooth. Each tooth was inserted into the universal testing machine and a shear force was applied to the bracket. The amount of shear force needed to debond the bracket was recorded.

FIG. 3B depicts the shear bond strengths of the four compositions of adhesive samples after bracket debonding. The control group exhibited a shear bond strength of about 14.15 Mpa which increased to about 16 Mpa for group 2 having propolis and adhesive resin. Group 3 having nanohydroxyapatite and adhesive had a shear bond strength of 14.4714 MPa. Group 4 having propolis, hydroxyapatite nanoparticles and adhesive resin exhibited a higher shear bond strength of about 15.7 MPa.

### Example 3: Percentage of Surface Hardness Change (% SH)

### Sample Preparation for % SH

The buccal surface of the teeth was screened to exclude teeth with demineralized areas, cracks, and cavitations. Sound enamel segments were sectioned from the crown using a diamond-separating disk. The included enamel samples were mounted in a cylindrical acrylic resin block and polished until they reached a mirror-like appearance using a polisher device (MetroServ 250 Grinder-Polisher, manufactured by Buehler, Hong Kong, China) and inspected under a microscope to ensure no dentin exposure.

### Baseline Measurement

The baseline surface hardness was measured using a Knoop indenter at 25 g for 10 seconds (MicroMet 6040 Hardness Tester, manufactured by Buehler, Shanghai, China). Three vertically separated indentations with 100 μm spacing between each indentation were measured to obtain the average baseline surface hardness. The value of the buccal enamel microhardness was measured, and any sample showing more or less than 20% of the total average was excluded. Knoop hardness values were randomly assigned to one of the four experimental groups (n=3).

### Demineralization

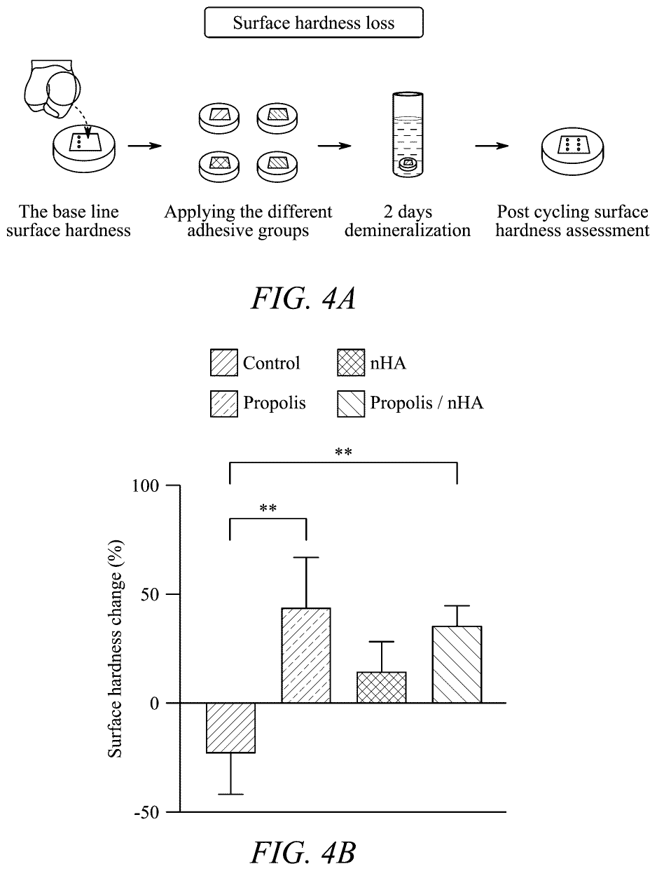

FIG. 4A illustrates the method for assessing the surface hardness loss. Each of the different adhesive compositions were placed directly over the prepared enamel samples without any etching or bonding to ease their removal later. For each group, the samples were immersed in demineralization solution (2.0 mmol/L Ca (NO₃)₂·4H₂O, 2.0 mmol/L NaH₂PO₄·2H₂O, 0.075 mmol/L acetate buffer, 0.02 ppm F at pH 4.7) for two days.

The enamel samples were then subjected to a post-surface hardness assessment. Adhesive materials were carefully removed from the enamel surface of the samples using a spoon excavator, after which all the samples were rinsed with distilled water and gently air-dried. Three new surface indentations were created using the same method as mentioned previously with 100 μm space between the initial and post-cycling indentions. The % SH for all the samples was measured with the following formula:

$$\% SH = \frac{Surface Hardness after pH cycling - Baseline Surface Hardness}{Baseline Surface Hardness} \times 100$$

The surface hardness % (% SH) values for all the samples including the control group is illustrated in FIG. 4B. The group having propolis and hydroxyapatite nanoparticles showed a surface hardness 60% higher than the control group adhesive lacking propolis and hydroxyapatite nanoparticles. The surface hardness of the group having propolis and hydroxyapatite nanoparticles is comparable to the surface hardness shown by the group having propolis and adhesive, which shows that propolis significantly contributes to the strength of the dental adhesives.

### Example 4: Antimicrobial Activity Against S. mutans

The antimicrobial properties were tested by preparing uniformly sized composite discs (10 mm in diameter and 2 mm in thickness) for use in colony-forming units (CFUs), MTT metabolic assays, and pH neutralization.

An S. mutans biofilm model was prepared. The mutans reference strain UA159 (ATCC, Manassas, VA, USA) was cultured for 24 hours in brain heart infusion (BHI) broth at 37° C. with 5% CO₂. The S. mutans culture was adjusted to an optical density of ~0.9 at 600 nm and then diluted in BHI broth supplemented with 2% sucrose to prepare the inoculum. Each sample was sterilized under ultraviolet light (Front Arch ultraviolet light box, Houston, USA) for 20 min. Then, the samples were placed in a 24-well plate filled with 1.5 mL of the inoculum for each well. The plate was incubated at 37° C. with 5% CO₂. After 24 hours, the media was replaced with 1.5 mL of fresh BHI broth supplemented with 2% sucrose and returned to the incubator. After 48 hours, the attached biofilm on each sample was assessed using colony-forming unit (CFU) counts and metabolic activity assays. The pH of the biofilm was also measured during the experiment.

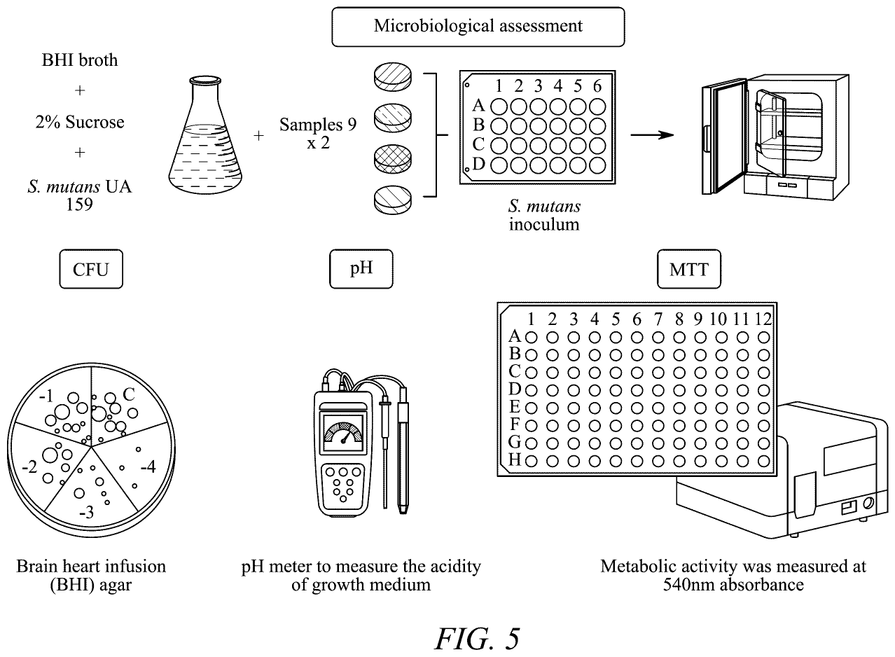

FIG. 5 illustrates a method for evaluation of the antimicrobial properties of the sample adhesives. The different groups of adhesive discs (n=7) were incubated for 48 h in the oven shown as described above. Each disc specimen with the attached biofilm was washed with phosphate-buffered saline, transported to a vial containing 2 ml of phosphate-buffered saline (PBS) and sonicated for 5 min using an orbital shaker to collect the biofilm. Then, the suspensions were serially diluted, cultured in brain heart infusion using a drop-counting technique, and incubated for 24 h. Each sample was plated in triplicate. A digital pH meter (PH20 pH tester, manufactured by Apera Instruments, Ohio, U.S.A.) was used to monitor the pH of the S. mutans biofilm (n=3) after 24 and 48 hours. The pH meter was periodically calibrated during the test by using commercial standard buffer solutions of pH 4, pH 7, and pH 10 that were kept at room temperature. The metabolic activity as shown in an MTT (3-[4,5-dimethylthiazol-2-yl]-2,5-diphenyltetrazolium bromide) test was measured at 540 nm absorbance. The results were calculated based on the number of colony-forming units (CFU) and the dilution factor, log (10). The results of CFU tests are expressed in logarithmic (log) values, where each log represents a ten-fold increase in the number of CFUs.

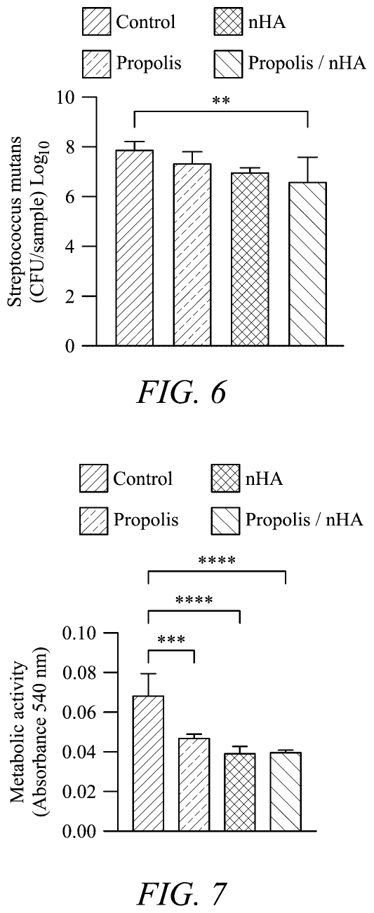

FIG. 6 illustrates the values of triplicated CFU tests for all the adhesive samples. The adhesive sample with propolis and hydroxyapatite nanoparticles shows an amplitude of about 7 based on a log₁₀ scale compared to the control adhesive sample having an amplitude of about 8 based on the log₁₀ scale, which indicates that the adhesive sample with propolis and hydroxyapatite nanoparticles shows about 10 times fewer colony forming units compared to the control sample and has anti-microbial properties which minimize the risk of white spot lesion formation after orthodontic treatments.

### Example 5: Metabolic Activity

As illustrated in FIG. 5, an MTT (3-[4,5-dimethylthiazol-2-yl]-2,5-diphenyltetrazolium bromide) test was used to measure S. mutans metabolic activity in the 48-hour biofilm. After incubating the different adhesive discs (n=7) with S. mutans for 48 h, each adhesive disc was washed with 1 ml of PBS and transported to a new 24-well plate containing 1 ml of tetrazolium dye in PBS (0.5 mg/mL) for 1 h to stain the attached biofilm. To solubilize the formazan crystals, the stained discs were transferred to a new 24-well plate (shown as plate with circles) containing 1 mL of dimethyl sulfoxide (DMSO), and then the plates were incubated for another 20 min at 37° C. After that, 200 L of the DMSO solution was collected from each disc to measure the absorbance at 540 nm (Bio-Rad xMark™, CA, USA). DMSO was used as a blank for the spectrophotometer. The mean standard deviations for all samples were calculated for Streptococcus mutans biofilm metabolic activity after about 48 hours of exposure to the Streptococcus mutans biofilm as illustrated in FIG. 7. Group 4 sample showed least value for mean standard deviation of about 0.04 when compared with groups 1, 2 and 3.

### Example 6: Acid-Neutralizing Activity

The digital pH meter was used to monitor the pH of the S. mutans biofilm (n=3) after 24 and 48 hours. The pH meter was periodically calibrated during the test by using commercial standard buffer solutions of pH 4, pH 7, and pH 10 that were kept at room temperature.

To summarize, the adhesive formulations comprising propolis and nano-hydroxyapatite significantly reduce plaque buildup and enamel demineralization associated with fixed orthodontic appliances. The compositions containing 5% Propolis, 2% nano-hydroxyapatite, and combinations thereof, enhance the antimicrobial properties of resin adhesives, potentially improving oral hygiene during orthodontic treatment.

A first embodiment describes the dental adhesive comprising propolis in an amount of about 4 wt. % to about 6 wt. %, calcium hydroxyapatite nanoparticles in an amount of about 1.5 wt. % to about 2.5 wt. % and the balance an orthodontic adhesive resin, wherein wt. % is based on a total weight of the dental adhesive.

In an aspect, the amount of the propolis is about 5 wt. %.

In an aspect, the amount of the calcium hydroxyapatite nanoparticles is about 2.0 wt. %.

In an aspect, the amount of the propolis is 5 wt. % and the amount of the calcium hydroxyapatite nanoparticles is about 2.0 wt. %.

In an aspect, the dental adhesive has a shear bond strength of about 15.7 MPa.

In an aspect, the surface hardness of the dental adhesive after two days of exposure to demineralization is greater by about 60% in comparison to a control dental adhesive.

In an aspect, the triplicated colony forming unit test for the dental adhesive has an amplitude of about 7 based on a log₁₀ scale compared to a control adhesive having an amplitude of about 8 based on the log₁₀ scale.

In an aspect, the dental adhesive has a mean standard deviation of about 0.04 of Streptococcus mutans biofilm metabolic activity after about 48 hours of exposure to the Streptococcus mutans biofilm.

A second embodiment describes a method of bonding an orthodontic device to a tooth comprising applying a layer of an orthodontic adhesive to the tooth and/or the orthodontic device, wherein the orthodontic adhesive comprises an propolis in an amount of about 4 wt. % to about 6 wt. %, calcium hydroxyapatite nanoparticles in an amount of from about 1.5 wt. % to about 2.5 wt. %, and the balance an orthodontic adhesive resin, wherein wt. % is based on a total weight of the dental adhesive and bonding the orthodontic device to the tooth by pressing the orthodontic device against the tooth with the orthodontic adhesive between the tooth and the orthodontic device.

In an aspect, the amount of the propolis is about 5 wt. %.

In an aspect, the amount of the calcium hydroxyapatite nanoparticles is about 2.0 wt. %.

In an aspect, the amount of the propolis is 5 wt. % and the amount of the calcium hydroxyapatite nanoparticles is about 2.0 wt. %.

In an aspect, the method comprises etching the tooth with phosphoric acid gel for about 30 seconds, washing the tooth with water for about 30 seconds, and air drying the tooth before applying the layer of an orthodontic adhesive to the tooth.

In an aspect, the method comprises curing, by a light emitting diode curing light, the orthodontic adhesive for 10 seconds after bonding the orthodontic device to the tooth.

In an aspect, the method comprises cleaning an excess orthodontic adhesive from the orthodontic device and tooth, and curing, by the light emitting diode curing light, the orthodontic adhesive for 40 seconds.

In an aspect, the orthodontic adhesive has a shear bond strength of about 15.7 MPa, a surface hardness of the orthodontic adhesive after two days of exposure to demineralization is greater by about 60% in comparison to a control orthodontic adhesive, a triplicated colony forming unit test of the cured orthodontic adhesive has an amplitude of about 7 based on a log₁₀ scale compared to the control orthodontic adhesive having an amplitude of about 8 based on the log₁₀ scale, and a mean standard deviation of about 0.04 of Streptococcus mutans biofilm metabolic activity after about 48 hours of exposure to the Streptococcus mutans biofilm.

A third embodiment describes a method of making a dental adhesive composition comprising mixing an amount of propolis of about 4 wt. % to about 6 wt. % and an amount of calcium hydroxyapatite nanoparticles in an amount of about 1.5 wt. % to about 2.5 wt. % into an orthodontic adhesive resin, wherein wt. % is based on a total weight of the dental adhesive composition.

In an aspect, the amount of propolis is about 5 wt. %.

In an aspect, the amount of calcium hydroxyapatite nanoparticles is about 2.0 wt. %.

In an aspect, the amount of propolis is 5 wt. % and the amount of calcium hydroxyapatite nanoparticles is about 2.0 wt. %.

The present disclosure is not limited to the specified combinations of propolis, hydroxyapatite nanoparticles and orthodontic adhesive resin as described herein. For example, the skilled artisan will appreciate that any combination of the components within the range as described, will certainly give results similar to those given by the specified combination. Also, numerous modifications and variations of the present disclosure are possible in light of the above teachings. It is therefore to be understood that within the scope of the appended claims, the invention may be practiced otherwise than as specifically described herein.

1. A dental adhesive, comprising:
propolis in an amount of about 4 wt. % to about 6 wt. %;
calcium hydroxyapatite nanoparticles in an amount of about 1.5 wt. % to about 2.5 wt. %; and
the balance an orthodontic adhesive resin, wherein wt. % is based on a total weight of the dental adhesive.

2. The dental adhesive of claim 1, wherein the amount of the propolis is about 5 wt. %, and the dental adhesive further comprises particles of a polyethylene wax in a propolis:wax particle weight ratio of from 1:0.9 to 1:0.5.

3. The dental adhesive of claim 1, wherein the amount of the calcium hydroxyapatite nanoparticles is about 2.0 wt. %.

4. The dental adhesive of claim 1, wherein:
the amount of the propolis is about 5 wt. %; and
the amount of the calcium hydroxyapatite nanoparticles is about 2.0 wt. %.

5. The dental adhesive of claim 4, having a shear bond strength of about 15.7 MPa.

6. The dental adhesive of claim 4, wherein:
a surface hardness of the dental adhesive after two days of exposure to demineralization is greater by about 60% in comparison to a control dental adhesive.

7. The dental adhesive of claim 4, wherein:
a triplicated colony forming unit test has an amplitude of about 7 based on a log₁₀ scale compared to a control adhesive having an amplitude of about 8 based on the log₁₀ scale.

8. The dental adhesive of claim 4, wherein the dental adhesive has a mean standard deviation of about 0.04 of Streptococcus mutans biofilm metabolic activity after about 48 hours of exposure to the Streptococcus mutans biofilm.

9. A method of bonding an orthodontic device to a tooth comprising:
applying a layer of an orthodontic adhesive to the tooth and/or the orthodontic device, wherein the orthodontic adhesive comprises propolis in an amount of about 4 wt. % to about 6 wt. %, calcium hydroxyapatite nanoparticles in an amount of from about 1.5 wt. % to about 2.5 wt. %; and the balance an orthodontic adhesive resin, wherein wt. % is based on a total weight of the dental adhesive; and
bonding the orthodontic device to the tooth by pressing the orthodontic device against the tooth with the orthodontic adhesive between the tooth and the orthodontic device.

10. The method of claim 9, wherein the amount of the propolis is about 5 wt. %.

11. The method of claim 9, wherein the amount of the calcium hydroxyapatite nanoparticles is about 2.0 wt. %.

12. The method of claim 9, wherein:
the amount of the propolis is 5 wt. %; and
the amount of the calcium hydroxyapatite nanoparticles is about 2.0 wt. %.

13. The method of claim 12, further comprising:
etching the tooth with phosphoric acid gel for about 30 seconds;
washing the tooth with water for about 30 seconds; and
air drying the tooth before applying the layer of an orthodontic adhesive to the tooth.

14. The method of claim 13, further comprising:
curing, by a light emitting diode curing light, the orthodontic adhesive for 10 seconds after bonding the orthodontic device to the tooth.

15. The method of claim 14, further comprising:
cleaning an excess orthodontic adhesive from the orthodontic device and tooth; and
curing, by the light emitting diode curing light, the orthodontic adhesive for 40 seconds.

16. The method of claim 15, wherein the orthodontic adhesive has a shear bond strength of about 15.7 MPa;
a surface hardness of the orthodontic adhesive after two days of exposure to demineralization is greater by about 60% in comparison to a control orthodontic adhesive;
a triplicated colony forming unit test of the cured orthodontic adhesive has an amplitude of about 7 based on a log₁₀ scale compared to the control orthodontic adhesive having an amplitude of about 8 based on the log₁₀ scale; and
a mean standard deviation of about 0.04 of Streptococcus mutans biofilm metabolic activity after about 48 hours of exposure to the Streptococcus mutans biofilm.

17. A method of making a dental adhesive composition, comprising:
mixing an amount of propolis of about 4 wt. % to about 6 wt. % and an amount of calcium hydroxyapatite nanoparticles in an amount of about 1.5 wt. % to about 2.5 wt. % into an orthodontic adhesive resin, wherein wt. % is based on a total weight of the dental adhesive composition.

18. The method of claim 16, wherein the amount of propolis is about 5 wt. %.

19. The method of claim 16, wherein the amount of calcium hydroxyapatite nanoparticles is about 2.0 wt. %.

20. The method of claim 16, wherein:
the amount of propolis is 5 wt. %; and
the amount of calcium hydroxyapatite nanoparticles is about 2.0 wt. %.
